(19) United States
(12) Patent Application Publication
(10) Pub. No.: US 2010/0096281 A1
(43) Pub. Date: Apr. 22, 2010

(54) APPARATUS FOR ACCESSING A MEDICAL PACKAGE

(76) Inventors: Joshua B. Stopek, Yalesville, CT (US); David Kirsch, Madison, CT (US)

Correspondence Address: Tyco Healthcare Group LP, 60 MIDDLETOWN AVENUE, NORTH HAVEN, CT, 06473, US

(21) Appl. No.: 12/528,567

(22) PCT Filed: Feb. 26, 2008
(86) PCT No.: PCT/US08/02457

§ 371 Date: Aug. 25, 2009

Related U.S. Application Data
(60) Provisional application No. 60/904,924, filed on Mar. 5, 2007.

(51) Int. Cl. A61B 17/06; B65D 17/42

(52) U.S. Cl. 206/63.3; 220/277

(57) ABSTRACT

The present disclosure describes an apparatus for accessing a medical device sealed within a package having a base configured and dimensioned to receive at least a portion of a sealed package containing a medical device and a cover adapted to overlie at least a portion of the sealed package adjacent the base. The cover includes a portal configured and dimensioned to receive a piercing structure therethrough for piercing the package and accessing the device sealed therein.

### CROSS-REFERENCE TO RELATED APPLICATION

The present application claims the benefit of and priority to U.S. Provisional Application Ser. No. 60/904,924, filed on Mar. 5, 2007, the entire disclosure of which is incorporated herein by reference.

### BACKGROUND

1. Technical Field

The present disclosure relates to an apparatus for medical packaging and more particularly, to an apparatus for accessing a medical device sealed within a package.

2. Background of Related Art

Medical professionals, e.g., surgeons, commonly treat medical devices with an agent prior to implantation. For example, sutures may be treated with a lubricant, adhesive, coating material or drug to improve certain physical characteristics of the suture like tensile strength, ease of sliding, knot tying or repositioning, and antimicrobial activity.

Often the medical professional will withdraw the medical device from the sterile packaging and then apply the agent using a variety of surgical utensils including a sharps device. Not only does withdrawing the device from the package decrease sterility, but application of the agent with the use of a sharps device may result in trauma to the medical professional. Moreover, this process of applying an agent to a medical device outside of the original package is time consuming and possibly inaccurate due to the likelihood of losing some amount of the agent to run-off.

Therefore, the present disclosure describes an apparatus for accessing a medical device sealed within a package for delivery of an agent between the outside of the apparatus and the medical device contained in the package. The apparatus allows the medical professional to efficiently dispense an agent to the medical device while preserving sterility and reducing waste of the agent.

### SUMMARY

The present disclosure describes an apparatus for accessing a medical device sealed within a package. The apparatus includes a base configured and dimensioned to receive at least a portion of a sealed package containing a medical device and a cover adapted to overlie at least a portion of the sealed package adjacent the base. The cover includes a portal which is positioned adjacent a predetermined location on the package and configured and dimensioned to receive a piercing structure therethrough for piercing the package and accessing the device sealed therein. In embodiments, the portal includes a piercing structure defined therein for piercing the package and accessing the device sealed therein. In other embodiments, the piercing structure is separate from the portal. Methods of accessing a medical device sealed within a package are also disclosed.

### BRIEF DESCRIPTION OF THE DRAWINGS

Various embodiments are described herein with reference to the drawings wherein:

FIG. 1 is perspective view of an apparatus in an open position as disclosed herein.

FIG. 2 is side view of an apparatus in a closed position as disclosed herein.

FIGS. 3A-3D are enlarged side views of mechanically engaging structures as disclosed herein.

FIG. 4 is perspective view of an apparatus in an open position as disclosed herein.

FIG. 5 is perspective view of an apparatus in an open position as disclosed herein.

### DETAILED DESCRIPTION OF PREFERRED EMBODIMENTS

An apparatus as described herein includes a base and a cover. The base is configured and dimensioned to receive at least a portion of a package containing a medical device. The cover is adapted to overlie at least a portion of the package adjacent the base and includes a portal defined therethrough. The portal may be positioned adjacent a predetermined location on the package and may be configured and dimensioned to receive a piercing structure therethrough for piercing the package and accessing the medical device contained therein.

It is envisioned that the base and the cover may be configured and dimensioned to any size and shape, including rectangular, triangular, circular and hexagonal shapes. It is further envisioned that the base and the cover do not have to be configured and dimensioned symmetrically. Rather, the base and cover may be configured and dimensioned into different sizes and shapes which are not symmetrical.

In embodiments, the apparatus is a monolithic structure wherein the base and the cover are made from one piece of material. In other embodiments, the apparatus may also include a base and a cover made from more than one piece of material thereby forming a structure which is not monolithic. The apparatus may be formed from any polymeric, ceramic, thermoplastic, metallic, natural, or synthetic materials suitable for accessing a medical device sealed within a package. Although any natural or synthetic polymeric material may be used to form the apparatus, some non-limiting examples include polymers, copolymers, homopolymers, block copolymers, and random copolymers including materials such as polyethylene, polypropylene, polycarbonates, polyesters, polycaprolactone, polyethylene terphthalate, and polysiloxanes.

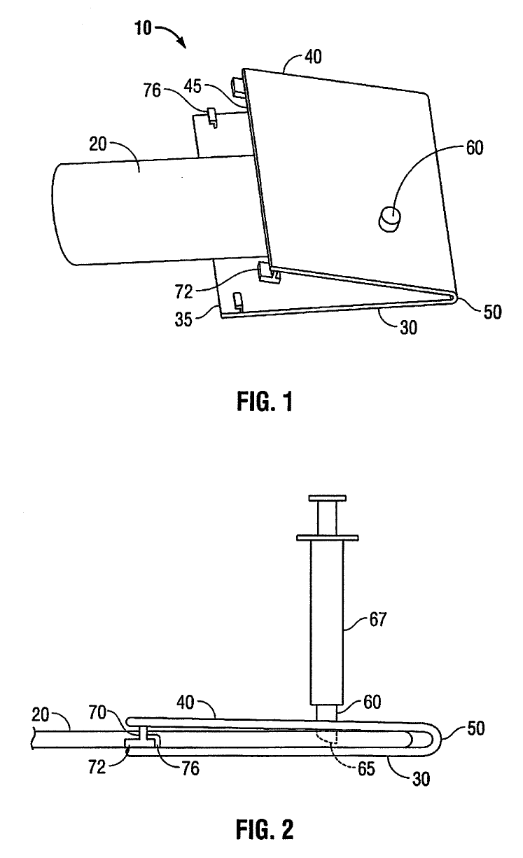

An apparatus which is a monolithic structure includes a base and cover that meet at one end of the apparatus and form a living hinge. A “living hinge” is defined as single piece of material having a natural biasing ability to pivot in one or more directions. In forming a living hinge, the apparatus described herein includes a base and a cover which are able to pivot from an open position (See FIG. 1) to a closed position (See FIG. 2) allowing a sealed package to enter and exit the apparatus.

An apparatus which is not a monolithic structure includes a base and a cover made from more than one piece of material which is connected by a hinge member (See. FIG. 5). The hinge member allows the base and cover to pivot from an open position to a closed position allowing a sealed package to enter and exit the apparatus as required.

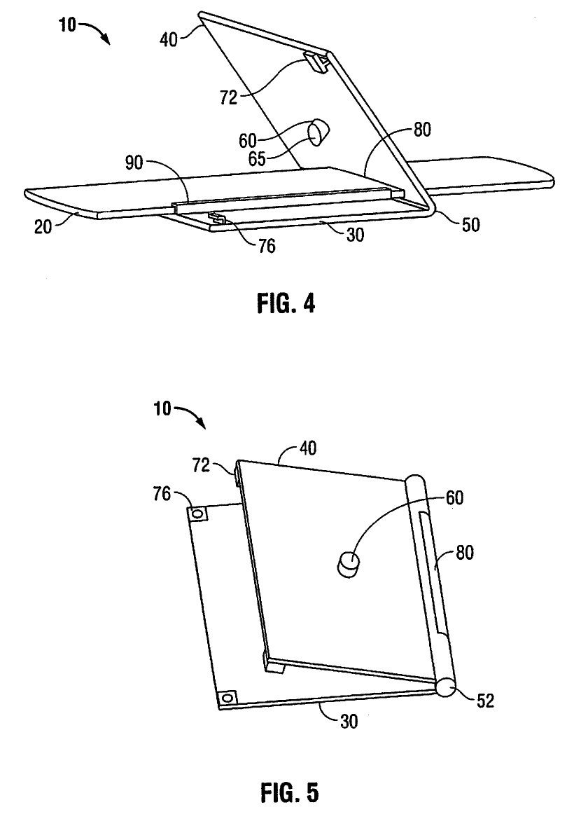

The cover includes a portal defined therethrough which is configured and dimensioned to receive a piercing structure therethrough for piercing the package and accessing the medical device sealed therein. In some embodiments, the portal may be a guide for receiving a separate piercing structure prior to accessing the sealed package (See FIG. 1). In some embodiments, the portal may include a self-sealing septum which requires a sharps device to penetrate the portal. In still other embodiments, the portal may include the piercing structure (See FIG. 4) capable of piercing the sealed package when the apparatus is in the closed position.

The portal is designed to allow the user of the apparatus to pass at least one agent between the medical device contained in the package and the outside of the apparatus. In some embodiments, the portal receives a delivery device containing a piercing structure and an agent which penetrates the sealed package and delivers the agent into the package to the medical device contained therein. In some embodiments, the delivery device can be used to withdraw fluids in which the medical device may be stored within the package.

The apparatus may be used to pass an agent between the inside of the package and the outside of the apparatus. The agent may be passed through the apparatus as a solid, liquid, semi-solid, gas, or any combination thereof. The at least one agent may be selected from any bioactive and/or non-bioactive agent suitable for combination with the medical device. Suitable agents include, but are not limited to, drugs, such as antiseptics, anesthetics, muscle relaxants, antihistamines, decongestants, antimicrobial agents, anti-viral agents, anti-fungal agents, antimalarials, amebicides, antituberculosal agents, antiretroviral agents, leprostatics, antiprotazoals, antihelmitics, antibacterial agents, steroids, hematopoietic agents, antiplatelet agents, anticoagulants, coagulants, thrombolytic agents, hemorrheologic agents, hemostatics, plasma expanders, hormones, sex hormones, uterine-active agents, bisphosphonates, antidiabetic agents, glucose-elevating agents, growth hormones, thyroid hormones, inotropic agents, antiarrhythmic agents, calcium channel blockers, vasodilators, sympatholytics, antihyperlipidemic agents, vasopressors, angiotensin antagonists, sclerosing agents, anti-impotence agents, urinary alkanizers, urinary acidifiers, anticholinergics, diuretics, bronchodilators, surfactants, antidepressants, antipsychotics, antianxiety agents, sedatives, hypnotics, barbiturates, antiemetic agents, analgesics, stimulants, anticonvulsants, antiparkinson agents, proton pump inhibitors, H₂-antagonists, antispasmodics, laxatives, antidiarrheals, antiflatulents, digestive enzymes, gallstone solubilizing agents, antihypertensive agents, cholesterol-lowering agents, radiopaque agents, immune globulins, monoclonal antibodies, antibodies, antitoxins, antivenins, immunologic agents, anti-inflammatory agents, antineoplastic agents, alkylating agents, antimetabolites, antimitotic agents, radiopharmaceuticals, vitamins, herbs, trace elements, amino acids, enzymes, chelating agents, immunomodulatory agents and immunosuppressive agents; coating materials such as lubricants, and non-bioabsorbable substances such as silicone, beeswax, or polytetrafluoroethylene, as well as absorbable substances such as collagen, chitosan, chitin, carboxymethylcellulose, and homopolymers and/or copolymers of polyalkylene glycols, and higher fatty acids or salts or esters thereof, glycolic acid, a glycolide, lactic acid, a lactide, p-dioxanone, valerolactone and other lactones derived from linear aliphatic hydroxycarboxylic acids, α-hydroxybutyric acid, ethylene carbonate, ethylene oxide, propylene oxide, propylene carbonate, malic acid ester lactones, succinic acid, adipic acid and other linear aliphatic dicarboxylic acids, and linear aliphatic diols such as butanediol and hexanediol; wound healing agents; adhesives; sealants; blood products; blood components; preservatives; colorants; dyes; ultraviolet absorbers; ultraviolet stabilizers; photochromic agents; anti-adhesives; proteins; polysaccharides; peptides; genetic material; viral vectors; nucleic acids; nucleotides; plasmids; lymphokines; radioactive agents; metals; alloys; salts; growth factors; growth factor antagonists; cells; hydrophobic agents; hydrophilic agents; immunological agents; anti-colonization agents; diagnostic agents; imaging agents; cross-linking agents; and diluents, such as water, saline, dextrose. Of course any combination of these agents may also be passed to the medical device contained in the package.

The sealed package may be made from any material suitable for forming packaging for a medical device. Some non-limiting examples include, foil-packs, Tyvek® bags, paper or plastic retainers and the like. The packages include any medical device or implant known to those skilled in the art. In embodiments, the medical device is selected from the group consisting of sutures, meshes, patches, slings, staples, anchors, pins, threads, adhesion barriers, and combinations thereof.

Referring now to FIGS. 1-5 there is shown an apparatus 10 for accessing a medical device within a package 20 having a proximal and a distal end. The term “proximal” will refer to the end of apparatus 10 which is closer to where base 30 and cover 40 meet, while the term “distal” will refer to the end which is further from where base 30 and cover 40 meet.

Turning now to FIG. 1, apparatus 10 is shown in an open position wherein the distal ends of base 30 and cover 40 are separated thereby allowing package 20 containing a medical device to be introduced into apparatus 10. Base 30 and cover 40 meet at the proximal end of apparatus 10 to form a living hinge 50 thereby allowing base 30 and cover 40 to pivot away from each other in the open position shown. Living hinge 50 also allows base 30 and cover 40 to pivot toward each other to form a closed position. (See FIG. 2) Cover 40 includes portal 60 which is shown as a guide for receiving a delivery device (not shown). Optionally, portal 60 may include a means for locking the delivery device into place, e.g., threads.

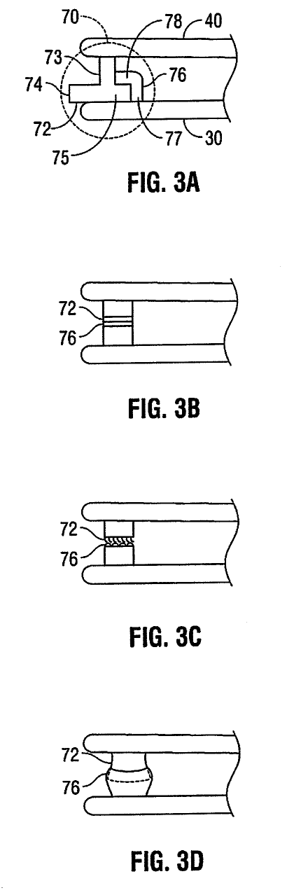

In addition, apparatus 10 further includes at least one mechanically engaging structure 70 which includes a first locking member 72 and a second locking member 76. It is envisioned that mechanically engaging structure 70 may include any type of first and second locking members 72, 76 capable of interacting with one another to lock base 30 and cover 40 in a closed position. As shown in FIGS. 3A through 3D, some non-limiting examples of suitable locking members include magnets, Velcro, snaps, interlocking devices and combinations thereof.

As further shown in FIG. 1, first locking member 72 is positioned adjacent a leading edge 45 of cover 40 and second locking member 76 is positioned adjacent a leading edge 35 of base 30. It is envisioned that the mechanically interlocking structure 70 may positioned anywhere along the perimeter of base 30 and cover 40 in a manner which allows the first and second locking members 72, 76 to engage one another.

In FIG. 2, package 20 is positioned within apparatus 10 in a closed position. Delivery device 67 is positioned within portal 60. Piercing structure 65 has accessed sealed package 20. In embodiments, piercing structure 65 is incorporated into portal 60 to access package 20. In some embodiments, piercing structure 65 is attached to delivery device 67 to access package 20 and portal 60. Base 30 and cover 40 meet at the proximal end of apparatus 10 to form a living hinge 50 which allows base 30 and cover 40 to pivot into vertical registration with one another to facilitate mechanical engagement. Upon pivoting cover 40 into vertical registration with base 30, first locking member 72 of cover 40 engages second locking member 76 of base 30 to maintain apparatus 10 in the closed position.

In embodiments as shown in FIG. 3A, mechanically engaging structure 70 includes first locking member 72 having a generally T-shape and second locking member 76 having a generally L-shape which are configured to mechanically interlock (See FIG. 3). The generally T-shaped first locking member 72 includes first arm 73 extending relatively downward from cover 40, forward flange 74 and trailing flange 75. The generally L-shaped second locking member 76 includes second arm 77 extending relatively upward from base 30 and edge 78 for engaging trailing flange 75 of first locking member 72.

It is envisioned that as cover 40 comes in vertical registration with base 30, first locking member 72 is passed over second locking member 76 forcing first locking member 72 to pivot slightly, allowing trailing flange 75 of first locking member 72 to become engaged by edge 78 of the second locking member 76. To release cover 40 from base 30, an upward force may be applied to forward flange 74 to force first locking member 72 to pivot slightly releasing trailing flange 75 from engagement with edge 78 of second locking member 76. The disengagement of first locking member 72 and second locking member 76 allows the bias of living hinge 50 to force base 30 and cover 40 to pivot away from each other into an open position.

FIGS. 3B-3D display other embodiments wherein first locking member 72 and second locking member 76 mechanically engage with the use of magnets, Velcro or snaps, respectively. Of course a combination of these locking members may also be used in maintaining apparatus 10 in the closed position.

Turning now to FIG. 4, apparatus 10 is shown in an open position including base 30 and cover 40 with portal 60 defined therethrough. In some embodiments, portal 60 includes piercing structure 65 for piercing sealed package 20 and accessing the medical device contained therein. Piercing structure 65 may be made from any polymeric or metallic material capable of penetrating package 20 to access the medical device.

In some embodiments, apparatus 10 further includes aperture 80 which is positioned along at least a portion of the proximal end of apparatus 10. Aperture 80 is configured and dimensioned to allow at least a portion of sealed package 20 to pass therethrough, as shown in FIG. 4. Package 20 is then able to enter the distal end of apparatus 10, pass through aperture 80 and exit near the proximal end of apparatus 10. Aperture 80 allows apparatus 10 to accommodate more of package 20, thereby allowing portal 60 to be positioned adjacent a larger portion of sealed package 20. Aperture 80 may be positioned adjacent living hinge 50. Alternatively, aperture 80 may be defined through living hinge 50.

In some embodiments, apparatus 10 further includes at least one guide member 90. Guide member 90 may be positioned on base 30 and/or cover 40 to assist the user of apparatus 10 in guiding sealed package 20 into apparatus 10. As shown, guiding member 90 is positioned generally perpendicular to living hinge 50 on base 30, however it is envisioned that guiding member 90 may be positioned in any pattern, design, or angle on apparatus 10.

It is further envisioned that apparatus 10 may receive sealed package 20 at any angle relative to the hinge portion of apparatus 10. Although shown in FIGS. 1-5 entering apparatus 10 perpendicular to living hinge 50, sealed package 20 may optionally enter apparatus 10 at an acute angle relative to living hinge 50. Alternatively, sealed package 20 may enter apparatus 10 in a manner parallel to living hinge 50.

In FIG. 5, apparatus 10 is not a monolithic structure and includes hinge member 52 which connects base 30 to cover 40 and allows base 30 and cover 40 to pivot into vertical registration with one another and facilitate mechanical engagement. In some embodiments, hinge member 52 may include a spring (not shown) or display a natural bias for remaining in the open position.

The apparatus described herein may be used to access a medical device sealed within a package. The method of use begins with the step of inserting a sealed package into an apparatus having a base configured and dimensioned to receive at least a portion of the package. The method continues with the steps of positioning a cover having a portal defined therethrough to overlie at least a portion of the sealed package adjacent the base and piercing the package with a piercing structure defined within the portal.

It should, of course, be understood that combinations of any of the embodiments described herein may be used to form an apparatus for accessing a medical device sealed within a package. It will be further understood that various modifications may be made to the embodiments disclosed herein. Therefore, the above description should not be construed as limiting, but merely as an exemplification of preferred embodiments. Those skilled in the art will envision other modifications within the scope and spirit of this disclosure. Various modifications and variations of the coated filaments and uses thereof will be apparent to those skilled in the art from the foregoing detailed description. Such modifications and variations are intended to come within the scope of the following claims.

What is claimed is:

1. An apparatus for accessing a medical device sealed within a package, said apparatus comprising:
a base configured and dimensioned to receive at least a portion of a sealed package containing a medical device,
a cover adapted to overlie at least a portion of said sealed package adjacent said base, said cover including a portal positioned adjacent a predetermined location on said package portion configured and dimensioned to receive a piercing structure therethrough for piercing said package and accessing said device sealed therein.

2. The apparatus of claim 1 wherein the apparatus is a monolithic structure.

3. The apparatus of claim 1 wherein the sealed package is a foil pack.

4. The apparatus of claim 1 wherein the portal is a self-sealing septum.

5. The apparatus of claim 1 further comprising a piercing structure.

6. The apparatus of claim 1 further comprising a mechanically engaging structure.

7. The apparatus of claim 6 wherein the mechanically engaging structure comprises a first locking member and a second locking member.

8. The apparatus of claim 7 wherein the first and second locking members are selected from the group consisting of magnets, snaps, rivets, straps, Velcro, interlocking devices and combinations thereof.

9. The apparatus of claim 7 wherein the first locking member is a generally T-shaped flange.

10. The apparatus of claim 9 wherein the generally T-shaped flange comprises an arm, a forward flange and a trailing flange for engaging the second locking member.

11. The apparatus of claim 7 wherein the second locking member comprises a generally L-shaped flange.

12. The apparatus of claim 11 wherein the generally L-shaped flange comprises an arm and edge for engaging the first locking member.

13. An apparatus for accessing a medical device sealed within a package, said apparatus comprising:
a base configured and dimensioned to receive at least a portion of a sealed package containing a medical device,
a cover adapted to overlie at least a portion of said sealed package adjacent said base, said cover including a portal positioned therethrough containing a piercing structure defined therein for piercing said package and accessing said device sealed therein.

14. The apparatus of claim 13 wherein the apparatus is a monolithic structure.

15. The apparatus of claim 13 wherein the sealed package is a foil pack.

16. The apparatus of claim 13 wherein the medical device is a suture.

17. The apparatus of claim 13 wherein the portal is a self-sealing septum.

18. The apparatus of claim 13 further comprising a mechanically engaging structure.

19. The apparatus of claim 18 wherein the mechanically engaging structure comprises a first locking member and a second locking member.

20. The apparatus of claim 19 wherein the first and second locking members are selected from the group consisting of magnets, snaps, rivets, straps, Velcro, interlocking devices and combinations thereof.

21. The apparatus of claim 19 wherein the first locking member is a generally T-shaped flange.

22. The apparatus of claim 21 wherein the generally T-shaped flange comprises an arm, a forward flange and a trailing flange for engaging the second locking member.

23. The apparatus of claim 19 wherein the second locking member comprises a generally L-shaped flange.

24. The apparatus of claim 23 wherein the generally L-shaped flange comprises an arm and edge for engaging the first locking member.

25. A method of accessing a medical device sealed within a package comprising the steps of:
inserting a sealed package within an apparatus having a base configured and dimensioned to receive at least a portion of the package,
positioning a cover to overlie at least a portion of said sealed package adjacent said base, said cover including a portal defined therethrough
piercing said package with a piercing structure defined within said portal.
